(19) United States
(12) Patent Application Publication
(10) Pub. No.: US 2005/0061329 A1
(43) Pub. Date: Mar. 24, 2005

(54) Catheter for intrafallopian contraceptive delivery

(75) Inventors: Quang Tran, Redwood City, CA (US); Mimi Nguyen, Foster City, CA (US); Rosendo Aguilar, Newark, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(73) Assignee: Conceptus, Inc., San Carlos, CA (US)

(21) Appl. No.: 10/665,973

(22) Filed: Sep. 18, 2003

(51) Int. Cl. A61F 6/06

(52) U.S. Cl. 128/831

(57) ABSTRACT

Catheters for intrafallopian contraceptive delivery generally improve the ease, speed, and reliability with which a contraceptive device can be deployed transcervically into an ostium of a fallopian tube. One or more coils disposed along the catheter near its distal end enhance the ability of the catheter through a tortuous lumen, such as a fallopian tube, without significant kinking or narrowing of the inner diameter of the catheter. A coil also helps maintain the inner diameter of the catheter when it elongates during removal, thus facilitating delivery of a contraceptive device from the catheter. A variable-flexibility distal portion in some embodiments further enhances maneuverability and pushability.

### BACKGROUND OF THE INVENTION

The present invention generally relates to medical devices for contraception and/or sterilization. More specifically, the invention relates to catheters for intrafallopian delivery of contraceptive devices.

While the theoretical effectiveness of existing non-surgical contraceptive techniques, including barrier methods and hormonal therapies, is well established, the actual effectiveness of most known methods is disappointing. One reason for these disappointing results is that many of the presently available methods for inhibiting pregnancy without surgery depend upon significant user involvement. Non-compliance typically results in quite high rates of failure, and overcoming user non-compliance to improve overall efficacy has proven quite difficult.

One form of long term contraception which is less susceptible to user non-compliance is the intrauterine device (IUD). IUDs have been found to have higher rates of reliability and are effective for a longer period of time than most other commercially available contraceptives. Unfortunately, IUDs are also associated with serious infectious complications. For this reason, the use of IUDs in the United States has decreased dramatically. Additionally, IUDs are subject to unplanned expulsion and must be removed due to excessive pain or bleeding in a significant percentage of cases, further reducing acceptance of the IUD as a method of inhibiting pregnancy.

Traditional methods for permanent sterilization include fallopian tube ligation and vasectomy. These methods are invasive, surgical procedures, which are undesirable to some people and not available to many people in the world.

One alternative to conventional contraceptive measures is to transcervically introduce a resilient coil into a fallopian tube to inhibit conception. Devices, systems and methods for such a contraceptive approach have been described in various patents and patent applications assigned to the present assignee. For example, PCT Patent Application No. 99/15116 and U.S. patent application Ser. No. 09/644,277, the full disclosures of which are incorporated herein by reference, describe devices which are transcervically inserted into an ostium of a fallopian tube (a “tubal ostium”) and mechanically anchored within the fallopian tube. The devices described in these applications may promote a tissue ingrowth network to provide long term conception and/or permanent sterilization without the need for surgical procedures, and should avoid the risks of increased bleeding, pain, and infection associated with intrauterine devices.

While the recently proposed intrafallopian contraceptive devices represent a significant advancement in the art, still further improvements would be desirable. Specifically, it is sometimes difficult with currently available catheters to maneuver the distal end of the catheter through the ostium into the fallopian tube to deliver a contraceptive device. Such a delivery catheter should be flexible enough to track the tortuous path of a fallopian tube, but should also have sufficient stiffness to provide pushability. Since the contraceptive device is typically carried at or near the distal end of the catheter, the catheter should ideally be configured to carry the contraceptive device without damaging it and allow for simple, accurate device delivery in the fallopian tube.

Many current catheters have variable flexibilities along their lengths, to allow for flexibility and pushability, but are either too flexible or too rigid at the distal end. Furthermore, many current catheters are made of braided material which tends to narrow down, or “neck,” when elongated or when extending through a curved passage. When a physician attempts to remove such a braided catheter over a contraceptive device, to leave the contraceptive device in place in the fallopian tube, the catheter may sometimes narrow down and catch on the device, making delivery difficult.

Therefore, it would generally be desirable to provide improved devices for inhibiting pregnancy. More specifically, it would be beneficial if these improved devices facilitated access to, and delivery of a contraceptive device within, a fallopian tube. It would be further beneficial if these improved access and deployment devices were suitable for a wide variety of physiological geometries, ideally without having to tailor the device, deployment system, or deployment method for specific individuals. At least some of these advantages are provided by the devices of the present invention.

### BRIEF SUMMARY OF THE INVENTION

The present invention generally provides an improved catheter for delivering contraceptive devices in fallopian tubes. Improved catheters generally enhance the ease, speed, and reliability with which a contraceptive device can be deployed transcervically into an ostium of a fallopian tube.

In one aspect, a catheter for delivering a contraceptive device within a fallopian tube includes an elongate tubular catheter body having a proximal portion adjacent a proximal end, a distal portion adjacent a distal end, at least one lumen, and at least one coil disposed along the catheter body nearer the distal end than the proximal end and encircling the lumen. Due to the presence of a coil in (or on) the catheter, throughout this application the terms “delivery catheter” and “coil catheter” are used interchangeably to mean any catheter described herein for delivering a contraceptive device.

Optionally, the distal portion of the catheter may have varying amounts of flexibility (or stiffness) along its length, typically being more flexible towards the distal end of the catheter than towards the proximal end. This variable-flexibility distal portion (or “distal tip”) facilitates maneuverability of the delivery catheter in a fallopian tube while also providing pushability. Meanwhile, the coil allows the distal portion to bend and/or elongate without significantly narrowing the inner diameter to impinge on the contraceptive device being delivered. Thus, a coil catheter may have several advantages over currently available catheters, which are typically braided.

In some embodiments, the distal portion of the catheter body has two or more layers, and the coil comprises one of the layers. For example, the catheter body may have an inner layer, a middle layer, and an outer layer, with the middle layer being the coil. The coil may be made of any suitable material, such as but not limited to a metal, such as Nitinol, stainless steel or titanium, or any suitable non-metallic material, such as a polymer. The coil may also have any suitable configuration, diameter and the like. In some embodiments, in fact, multiple coils may be used. In one embodiment, the pitch of the coil (i.e., the longitudinal distance along the catheter from one coil to the next adjacent coil) is 0.030 cm, though any other suitable pitch, shape or the like may be used.

As mentioned, the distal portion of the catheter body is typically more flexible towards the distal end than towards the proximal end. Often, the distal portion is relatively short relative to the overall length of the catheter. Thus, the coil catheter has a short distal tip with variable flexibility, the flexibility increasing distally. Although some currently available catheters have variable flexibilities, these usually occur along the entire length of the catheter, not in a relatively short distal portion. In some embodiments, to impart this variable flexibility, the distal portion includes two or more segments, with distal segments being progressively more flexible than proximal segments. In one embodiment, for example, the distal portion includes three segments which are increasingly flexible from proximal to distal. Different segments may be formed of different materials, different thicknesses of the same material, and/or the like. Such a variable-flexibility distal portion may prove advantageous in navigating a tortuous fallopian tube to deliver a contraceptive device.

In some embodiments, the distal end of the proximal portion of the catheter may overlap the proximal end of the distal portion of the catheter. For example, the proximal portion may overlap the coil and the inner layer of the distal portion. This overlapping area will enhance the connection between the proximal and distal portions.

Any layer, and sometimes multiple layers, may have a hydrophilic surface (or surfaces) or other friction-reducing surface(s) to enhance delivery of the catheter into a fallopian tube, delivery of a contraceptive device from the catheter, insertion over or removal of a guidewire, and/or the like. In some embodiments, for example, the innermost surface of the catheter and/or the outermost surface of the catheter may have a hydrophilic coating, such as silicone, MDX or the like. In any embodiment, such coating(s) may extend the entire length of the catheter or may extend along only part of the catheter.

Just as the coil may comprise any suitable material, so to may other layers or sections of the catheter be made of any suitable material. For example, in one embodiment an inner layer of the distal portion comprises Teflon®, and an outer layer comprises a polyurethane material. In some embodiments, the proximal portion of the catheter comprises a one-lumen tubular member of a material such as a polyether block amide. In many embodiments, the outer layer of the distal portion may include several different materials, the materials varying along the length of the distal portion to confer different flexibility (or stiffness) to different portions of the distal portion. In one embodiment, the outer layer of the distal portion comprises one or more types and/or thicknesses of a polyurethane material, such as Carbothane. Polyurethanes of different durometer readings may be used and/or different amounts or various numbers of layers of polyurethane(s) may be used to provide variable flexibility/stiffness along the distal portion. Alternatively, any other suitable materials and combinations may be used for making any layers or segments of the catheter. Typically, material(s) will be used for the distal portion of the catheter to give the distal portion increasing flexibility towards the distal end. There is no requirement, however, that multiple layers or multiple segments be used. Furthermore, the coil may be positioned in any suitable location or configuration, such as on an outer or inner surface of the distal portion, within any layer, between any two layers, or the like.

Catheters of the present invention may have any suitable length and configuration. Typically, the catheters may be from about 25 cm to about 70 cm in length, and more preferably between about 40 cm and about 60 cm, and even more preferably between about 43 cm and about 50 cm in length. The distal portion of a catheter may also have any suitable length, although in many embodiments the distal portion is relatively short compared to the overall length of the catheter. In one embodiment, for example, the distal portion has a length of between about 0.5 cm and about 3.0 cm, and preferably between about 1.2 cm and about 2.0 cm. The coil, too, may have any suitable length, but in some embodiments it measures between about 0.5 cm and about 4.0 cm, and preferably between about 1.6 cm and about 2.4 cm.

The proximal portion of the catheter may have any suitable configuration and may comprise any suitable material or combination of materials. In one embodiment, an inner diameter of the proximal portion of the catheter body is smaller near the distal end of the catheter body than near the proximal end—i.e., the inner diameter tapers from proximal to distal. This may facilitate passage of a guidewire or other instrument or wire into and through the proximal portion of the catheter. The proximal portion may comprise any suitable material or materials, such as a polyether block amide in one embodiment, and may be constructed from hydrophilic material or other friction reducing material. Alternatively, or additionally, the outer surface of the proximal portion may have a hydrophilic coating. In some embodiments, the proximal portion of the catheter body further includes at least one visualization marker near the distal portion for enhancing visualization of a proximal-most end of the distal portion. Such a visualization marker may include, but does not require, at least one radiopaque material.

In another aspect of the invention, a catheter for delivering a contraceptive device within a fallopian tube includes an elongate tubular catheter body having a proximal portion adjacent a proximal end, a distal portion adjacent a distal end, and at least one lumen, with the distal portion being more flexible towards the distal end than towards the proximal end. The catheter also includes at least one coil disposed along the catheter body nearer the distal end than the proximal end and encircling the lumen.

In another aspect, a catheter for delivering a contraceptive device within a fallopian tube includes an elongate tubular catheter body of between about 43 cm and about 50 cm, the catheter body having a proximal portion adjacent a proximal end, a distal portion of between about 1.2 cm and about 2.0 cm adjacent a distal end, at least one lumen, with the distal portion being more flexible towards the distal end than towards the proximal end. Again, the catheter also includes at least one coil disposed along the catheter body nearer the distal end than the proximal end and encircling the lumen.

In yet another aspect, a system for delivering a contraceptive device within a fallopian tube includes a catheter as described above, a contraceptive device releasably disposed at least partially within the lumen of the catheter near the distal portion, and a deployment member in detachable engagement with the contraceptive device for deploying the contraceptive device from the catheter.

Finally, in another aspect a method for making a catheter for delivery of a contraceptive device within a fallopian tube includes first forming a distal portion of the catheter by positioning a helical coil around an inner tubular member and placing at least one outer layer of material over the helical coil and the inner tubular member. The distal portion is then coupled with a proximal portion of the catheter. In some embodiments, coupling the proximal and distal portions involves overlapping a distal end of the proximal portion of the catheter with a proximal end of the distal portion of the catheter. Coupling the two portions may also involve heat welding the proximal portion to the distal portion.

Optionally, the method may further include coupling a first segment of the outer material with at least a second segment of the outer material. It may further involve coupling a third segment of the outer material with the second segment. In such embodiments, the first segment of the outer material has greater flexibility that the second segment, the second segment has greater flexibility than the third segment, and the third segment is coupled with the proximal portion of the catheter. Although such catheters may have any suitable lengths and configurations, in one embodiment the distal portion of the catheter is between about 1.2 cm and about 2.0 cm, the coil is between about 1.6 cm and about 2.4 cm, and the catheter is between about 43 cm and about 50 cm in length.

### BRIEF DESCRIPTION OF THE DRAWINGS

FIG. 1 illustrates the uterine and tubal anatomy for deployment of the contraceptive devices of the present invention.

FIG. 2 is a partial cut-away side view of a contraceptive system as described in U.S. patent application Ser. No. 09/644,277, previously incorporated by reference.

FIG. 3 illustrates the uterine and tubal anatomy with a catheter system in place for deployment of a contraceptive device.

FIG. 4 is a cross-sectional side view of a coil catheter according to one embodiment of the present invention.

FIG. 5 is an exploded side view of a coil catheter according to one embodiment of the present invention.

### DETAILED DESCRIPTION OF THE INVENTION

The present invention provides a delivery catheter (or “coil catheter”) for delivering a contraceptive device in a fallopian tube to inhibit pregnancy, typically for the long-term inhibition of pregnancy, and often providing permanent contraception or sterilization. By introducing such a contraceptive device into an ostium of a fallopian tube, the risks of unplanned expulsion, pelvic pain, and infectious complications may be significantly reduced. For further description of systems, devices and methods used with the coil catheter to provide contraception, reference may be made to U.S. patent application Ser. No. 09/644,277, which was previously incorporated by reference.

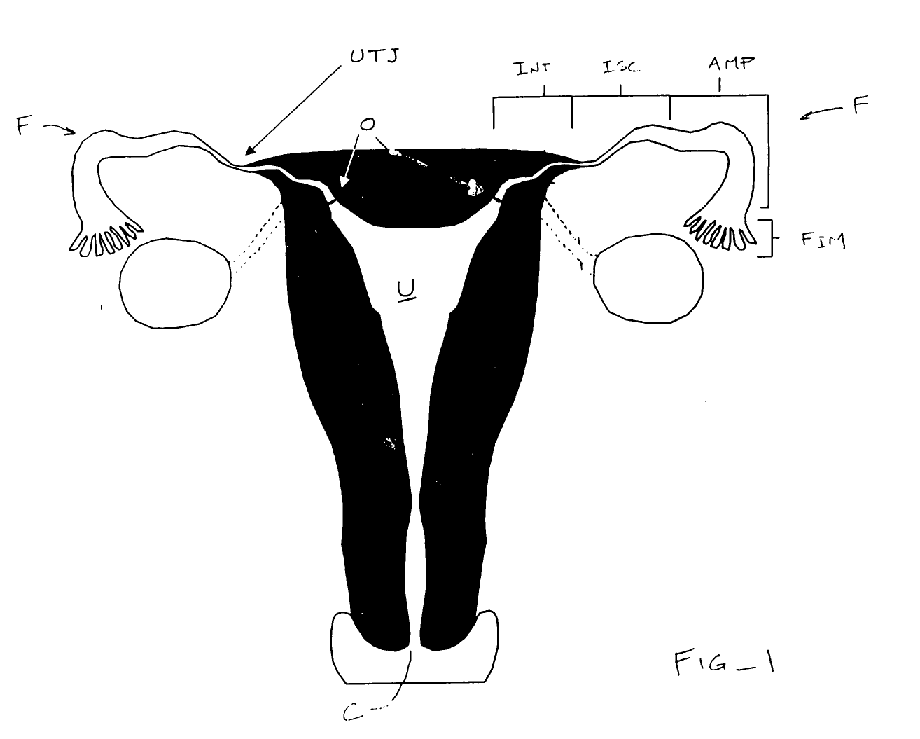

With reference now to FIG. 1, a diagram of a female reproductive tract is shown. As used herein, a structure is inserted “within a tubal ostium” whenever the structure is advanced from the uterus into (and optionally beyond) the tubal ostium, the uterotubal junction, and/or the fallopian tubes. Referring to FIG. 1, access to uterus U will generally be gained through cervix C. From within uterus U, fallopian tubes F are accessed via tubal ostia O.

Fallopian tubes F generally include three segments between ostium O and the fimbria FIM. Beginning adjacent uterus U, the intramural segment INT of fallopian tubes F are surrounded by the muscular uterine tissues. Beginning at uterotubal junction UTJ, fallopian tubes F extend beyond the uterine tissues and within the peritoneal cavity along an isthmic segment ISC, and then along an ampullary segment AMP.

In general, the ideal placement for intrafallopian contraceptive devices such as those described in U.S. patent application Ser. No. 09/644,277 is spanning the intramural INT to isthmic ISC portion of the fallopian tube. Where a radially expandable attachment mechanism such as an outer coil is included on the intrafallopian contraceptive device, that expandable or anchoring structure will preferably span the uterotubal junction UTJ. It should be noted that the uterotubal junction UTJ may be defined as the plane where the fallopian tube meets the peritoneal cavity. It should also be noted that the narrowest portion of the fallopian tube need not necessarily be located in the isthmic segment ISC, particularly once the contraceptive fallopian device (often having a radially expandable anchoring structure) is deployed therein. In fact, the effectively narrowest portion of the tube may be at or adjacent the uterotubal junction UTJ.

The exemplary contraceptive delivery catheter will preferably be able to accommodate a wide variety of anatomies. Two factors contribute to the importance of this variability: First, a wide variation may be observed between tubal anatomies of differing patients. Secondly, it can be quite difficult to determine and identify the specific tubal anatomy of a particular patient.

Generally, methods for delivery of a contraceptive device in which the delivery catheter of the present invention will be used involve positioning the distal end of the catheter at a desired location in a fallopian tube, freeing a contraceptive device from the catheter, and removing the catheter from the fallopian tube, leaving behind the contraceptive device. Again, further details of such methods are disclosed in U.S. patent application Ser. No. 09/644,277.

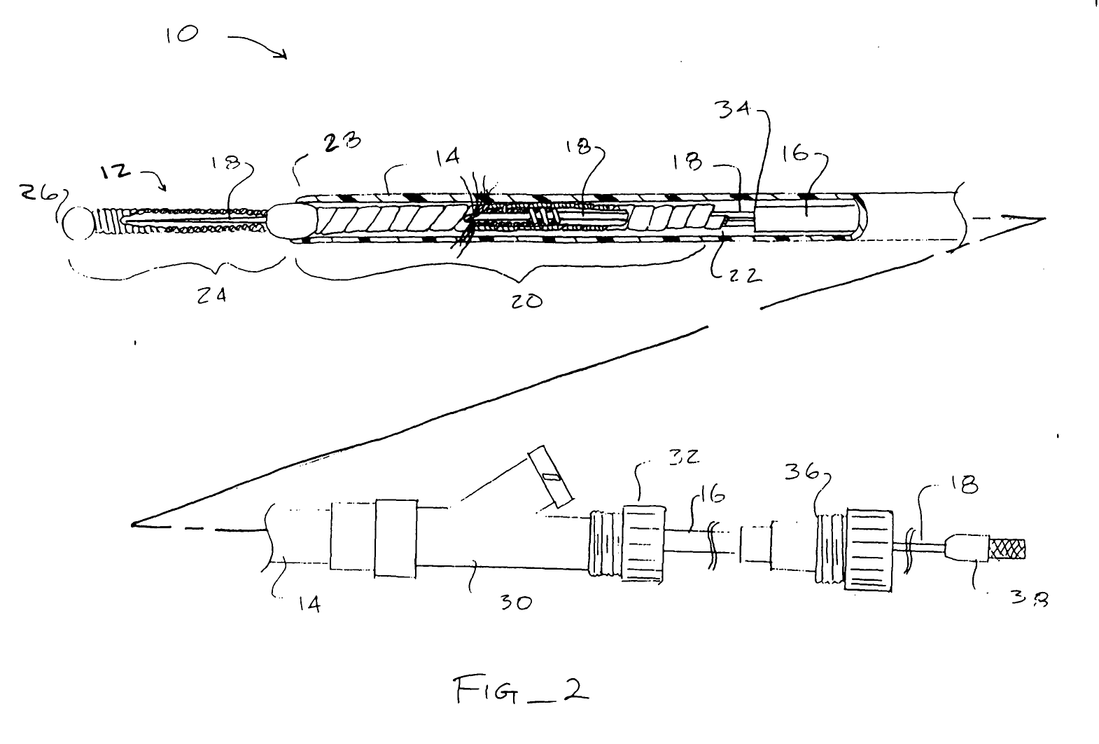

Referring now to FIG. 2, an exemplary contraceptive system 10 generally includes a contraceptive device 12, a delivery catheter 14 (referred to as a “sheath” in U.S. patent application Ser. No. 09/644,277) partially surrounding the contraceptive device, a release catheter 16, and a core shaft 18. Contraceptive device 12 generally has a proximal portion 20 adjacent a proximal end 22 (disposed within delivery catheter 14), and a distal portion 24 adjacent a distal end 26 (which are exposed beyond the distal end of delivery catheter 14). Distal portion 24 generally functions as a distal guidewire while system 10 is advanced within the tubal ostium. Proximal portion 20 includes a radially expandable structure which can be expanded after delivery catheter 14 is withdrawn so as to affix the contraceptive device in the deployed position.

As discussed above, the present invention provides improved delivery catheters for use with systems such as those shown in FIG. 2. Such catheters, however, may be used in any suitable contraceptive delivery system or in other catheter-based systems where it may be advantageous to have a catheter with a distal portion that does not constrict, narrow, or “neck down” when elongated or when navigating a curved lumen or passage. Therefore, despite the description of FIG. 2 below, delivery catheters of the present invention may be used with other devices or systems without departing from the scope of the invention.

Delivery catheter 14 is generally a tubular structure having a distal end 28 and extending proximally to a proximal housing 30. Delivery catheter 14 will generally have a length in a range from about 25 to about 70 cm, and will typically have an outer diameter in a range from about 0.020 inches to about 0.060 inches, one exemplary catheter having a length of about 46.5 cm and an outer diameter of about 0.04 inches. The inner diameter of delivery catheter 14 may be in a range from about 0.02 inches to about 0.05 inches, with an exemplary catheter having an inner diameter of about 0.033 inches. Proximal housing 30 includes a side arm with an injection port to allow infusion of fluids for patency checks, delivery of local anesthetic, or the like. Proximal housing 30 also includes a Touhy-Borst valve 32 releasably securing delivery catheter 14 to release catheter 16.

Release catheter 16 generally comprises a tube having a distal end 34 which releasably engages contraceptive device 12, and a proximal end adjacent a proximal fitting 36. Release catheter 16 will generally be longer than coil catheter 14, and fitting 36 will include another Touhy-Borst valve releasably securing release catheter 16 to core shaft 18. The release catheter length is sufficiently longer than the coil catheter 14 so that full retraction of the sheath exposes the distal end of the release catheter, thereby allowing the release of the expandable structure upon movement of the release catheter to be hysteroscopically monitored. It should be understood that the Touhy-Borst valve may be replaced by any coupling structure which inhibits axial and rotational movement between the coupled devices, such as a key-slot arrangement or the like.

In the exemplary embodiment, core shaft 18 comprises a resilient tapering structure extending from within distal portion 24 of contraceptive device 12 proximally through fitting 36 of release catheter 16 to a proximal handle 38. Core shaft 18 threadably engages contraceptive device 12 proximally of distal end 28 of delivery catheter 14 before deployment. In the exemplary embodiment, core shaft 18 and release catheter 16 transmit a wind-down torque onto an expandable structure of the contraceptive device so as to maintain the expandable structure in the small profile configuration. Hence, release catheter 16 relative to releasing core shaft 18 by actuating the Touhy-Borst valve of fitting 36 allows the expandable structure to be activated independently of movement of the surrounding sheath.

While exemplary contraceptive device 12 makes use of a radially expandable helical coil to help restrain the structure during tissue ingrowth, a wide variety of mechanical and other restraint mechanisms might be included. For example, alternative mechanical anchors might be attached to the device, such as resilient coils biased to form bends, loops, and/or other secondary shapes having enhanced cross-sections, slotted tubes, Malecot-type structures, radially expandable braids, stent-like devices, and the like. The mechanical structures may be resilient, plastically deformable, or the like, and suitable structures are described in more detail in, for example, PCT Publication No. WO 99/15116.

Still further device-restraint techniques might be employed, including thermal, chemical, adhesive, and the like. These techniques can be used to avoid expulsion by increasing friction between the device and the surrounding tissues, by imposing limited tissue damage to promote scar tissue formation, and/or by promoting tissue ingrowth into the device. Thermal techniques may include, for example, transmission of electrical or laser energy along contraceptive system 10. Resistive heating of contraceptive device 10 might be effected by applying an electrical potential across the device with conductors extending along delivery catheter 14 and release catheter 16, laser energy along an optical wave guide attached to core wire 18, or the like. Monopolar tissue desiccation might be effected via a large return electrode patch by energizing core wire 18 with radiofrequency energy, or an adhesive and/or caustic agent (such as a cyanoacrylate or silver nitrate) might be introduced via any of the lumens of the delivery system, via a dedicated lumen or structure, or the like. Biodegradable plugs and the like might also be included, and the retained structure may optionally comprise copper or other bioactive agents to help inhibit conception.

Tissue reaction to the retained contraceptive device 12 can help to provide long term contraception and/or sterilization. To promote conception inhibiting tissue reaction, device 12 will often include a tissue reaction material, the material often comprising fibers. The fibers may comprise a polyether, such as Dacron® polyethers, silk, nylon, or the like. The fibers may be in the form of a weave, a knit, a braid, a felt, or the like, or may comprise strands attached to the device body.

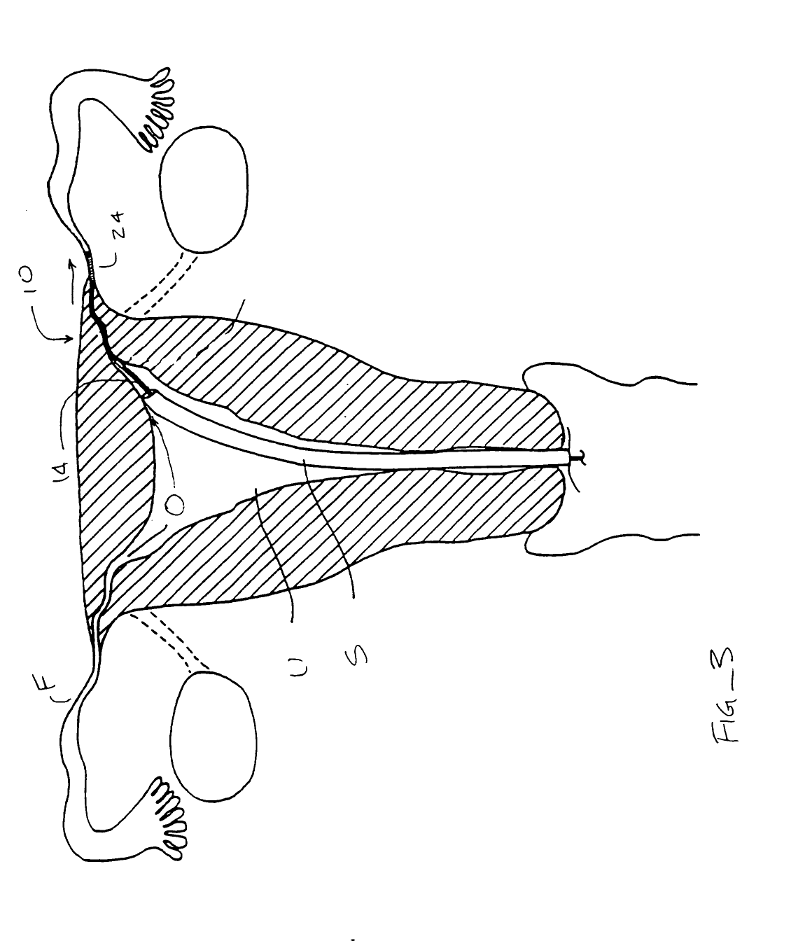

Referring now to FIG. 3, a contraceptive system as described above is shown in position for delivery of a contraceptive device. System 10 is introduced transcervically through uterus U, generally under optical direction. Using hysteroscope S the physician directs the distal end of the system toward ostium O of fallopian tube F. Alternatively, some or all of the procedure may be performed under any medical imaging modality, including fluoroscopy, sonography, computer tomography, or the like. Uterus U may be irrigated using scope S and/or a separate irrigation system. Once ostium O is located and the scope S is oriented toward the ostium, system 10 is advanced distally through the working lumen of the scope and through the ostium and into the fallopian tube using distal portion 24 of the contraceptive device as a guidewire, while the remainder of the contraceptive device remains covered by delivery catheter 14. Once delivery catheter 14 is used to position the distal portion 24 of the contraceptive device in a desired location of the fallopian tube F, the delivery catheter 14 is removed over the device to release the device and leave it in place in the fallopian tube F. Again, for further description of exemplary methods and systems which may make use of delivery catheters of the present invention, reference may be made to U.S. patent application Ser. No. 09/644,277.

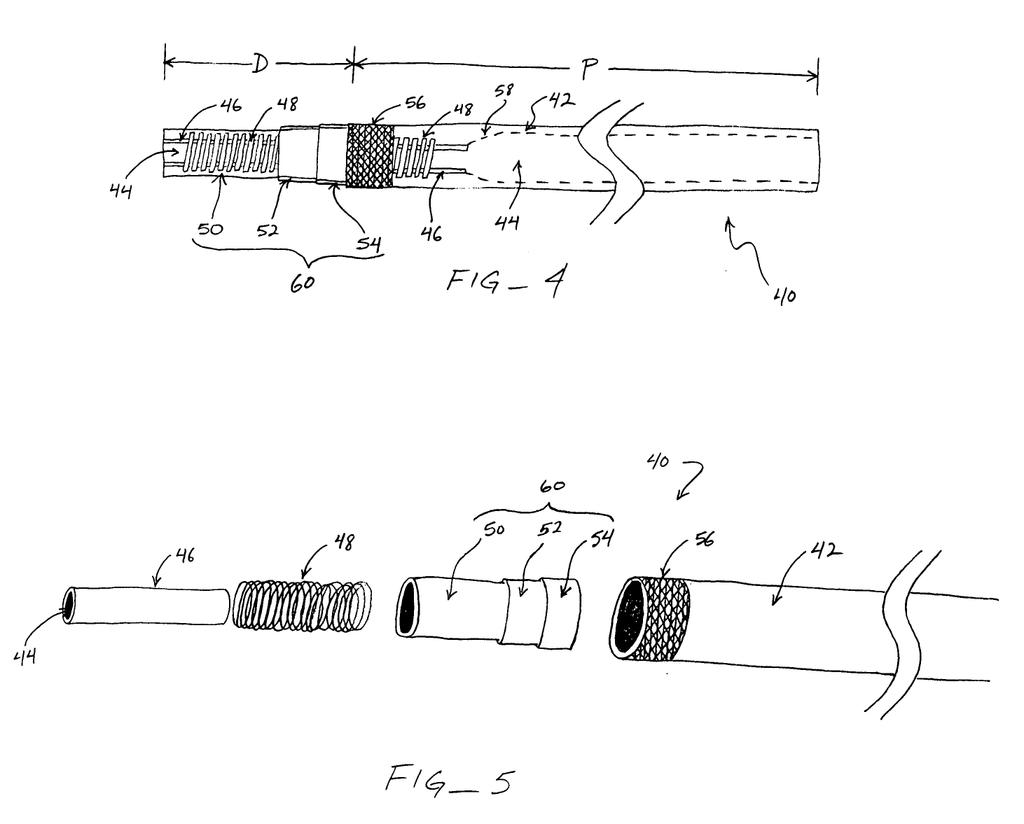

With reference now to FIG. 4, a coil catheter 40 for intrafallopian delivery of contraceptive devices suitably includes a proximal portion P and a distal portion D. In some embodiments, proximal portion P comprises a proximal catheter body 42, which is generally elongate and tubular, defining a lumen 44 and (optionally) having a marker 56 at or near its distal end to enable a user to more easily visualize the area where proximal portion P joins distal portion D. Distal portion D suitably includes a coil 50, or multiple coils, and one or more other layers within and/or around coil 50 and surrounding lumen 44. In the embodiment shown, an inner layer 46 is disposed within coil 48, and an outer layer 60 is disposed over coil 48. Outer layer 60, in turn, includes a proximal segment 54, a middle segment 52 and a distal segment 50. Although the following discussion focuses on the embodiment shown in FIG. 4, many other suitable configurations for coil catheter 40 are contemplated within the scope of the invention. For example, different combinations of materials, various placements of coil 48 and other features, alternative layering or segmenting of materials and the like may be used to achieve the desired effect without departing from the scope of the invention.

Most generally, coil catheter 40 is an elongate tubular member having a proximal end, a distal end, at least one lumen, and at least one coil disposed along catheter 40 nearer the distal end than the proximal end. Catheter 40 and coil 48 may be of any suitable length, diameter, shape or configuration and may be made of any suitable materials. In some embodiments, for example, catheter 40 has a total length of between about 25 cm and about 70 cm, and preferably between about 40 cm and about 60 cm, and even more preferably between about 43 cm and about 50 cm. Coil 48, in one embodiment, is between about 0.5 cm and about 3.0 cm, and preferably between about 1.2 cm and about 2.8 cm, and even more preferably between about 1.6 cm and about 2.4 cm. Generally, coil 48 enhances the maneuverability of catheter 40 by allowing distal portion D of catheter 40 to navigated curves or turns in a fallopian tube with relatively little kinking or narrowing of the inner diameter of catheter 40.

Distal portion D of catheter 40 may also have any suitable length and configuration, but in many embodiments distal portion D is a relatively short portion compared with the overall length of catheter 40. For example, in some embodiments distal portion D has a length of between about 0.5 cm and about 2.5 cm, and preferably between about 1.2 cm and about 2.0 cm, and more preferably between about 1.5 cm and about 1.7 cm. The distal portion D may include one or more layers or structures in addition to coil, such as inner layer 46 and outer layer 60. Coil 48 may be disposed in any suitable location within or on the surface of any such structures or layers of distal portion D. As shown in FIG. 4, coil 48 may also extend into proximal portion P in some embodiments.

In some embodiments, distal portion D of the catheter 40 has two or more layers, and coil 48 comprises one of the layers. For example, distal portion D may have inner layer 46, a middle layer, and outer layer 60, with coil 48 comprising the middle layer. Alternatively, coil 48 may be dispose between any two layers, within any layer, or on an outer or inner surface of any layer. Coil 48 may be made of any suitable material, such as but not limited to a metal, such as Nitinol®, stainless steel or titanium, or any suitable non-metallic material, such as a polymer. Coil 48 may also have any suitable configuration, diameter and the like. In some embodiments, in fact, multiple coils may be used. In one embodiment, the pitch of coil 48 (i.e., the longitudinal distance along the catheter from one coil to the next adjacent coil) is 0.030 cm, though any other suitable pitch, shape or the like may be used.

In the embodiment shown in FIG. 4, distal portion D includes inner layer 46, which defines lumen 44 within the distal portion D. Inner layer 46 may be made of any suitable material, such as but not limited to a friction-resistant material such as Teflon®, etched PTFE, a fluoropolymer, or the like. Outer layer 60 may also be fabricated from any material or combination of materials. In some embodiments, outer layer is made of one or more polyurethane materials. For example, a polyurethane such as Carbothane may be used. In one embodiment, a first polyurethane having a more flexible durometer rating (e.g., 73A) is used to make distal segment 50, a second polyurethane having a less flexible (stiffer) durometer rating (e.g., 55D) is used to make middle segment 52, and two layers of the less flexible polyurethane are used to make proximal segment 54. Of course, many other suitable materials and configurations are possible and are contemplated by the present invention. Generally, outer layer 60 is configured such that distal portion D is more flexible towards the distal end and stiffer towards the proximal end, thus enhancing both maneuverability and pushability. Again, many other possible configurations may be used, such as different materials for inner layer 46, a coil 48 with different tension towards the distal end, one continuous outer layer 60 of varying thickness and/or the like.

The proximal portion P of catheter 40 may have any suitable configuration and may comprise any suitable material or combination of materials. In one embodiment, the inner diameter of the proximal portion P of the catheter body is smaller near the distal end of the catheter body than near the proximal end—i.e., the inner diameter tapers from proximal to distal over at least one tapered region 58. This may facilitate passage of a guidewire or other instrument or wire into and through the proximal portion P. The proximal portion P may comprise any suitable material or materials, such as a polyether block amide in one embodiment, and may be constructed from hydrophilic material or other friction reducing material. Alternatively, or additionally, the outer surface of the proximal portion P may have a hydrophilic coating. In many embodiments, both proximal portion P and distal portion D are coated with a hydrophilic coating, such as silicone, MDX or any other suitable coating for reducing friction. In some embodiments, the proximal portion P further includes at least one visualization marker 56 near the distal end for enhancing visualization of the junction between the distal portion D and the proximal portion P. Visualization marker 56 may include, but does not require, at least one radiopaque material.

A mentioned previously, proximal portion P may overlap one or more components of distal portion D. Such an overlap may enhance connection of distal portion D with proximal portion P, thus making catheter 40 more durable. In the embodiment shown in FIG. 4, proximal portion overlaps part of coil 48 and inner layer 46, but any other configuration may be suitable and is contemplated within the scope of the invention.

Referring now to FIG. 5, catheters 40 as described above may be manufactured by any of a number of suitable methods. In one method, distal portion D is assembled by coupling inner layer 46 with coil 48 and disposing outer layer 60 over coil 48 and inner layer 46. Distal portion D may then be coupled with proximal portion P, and the two may be more permanently joined by heat welding, shrink wrapping and/or the like. In some embodiments, when distal portion D and proximal portion P are coupled, a hydrophilic or other friction-reducing coating may be deposited over the outer surface of catheter 40.

Optionally, a method for making catheter 40 may include coupling first segment 50 of outer layer 60 with at least a second segment 52 of outer layer 60. It may further involve coupling third segment 54 with second segment 52. As previously discussed, in such embodiments first segment 50 has greater flexibility that second segment 52, second segment 52 has greater flexibility than third segment 54, and third segment 54 is coupled with the proximal portion P of catheter 40.

While the exemplary embodiment of the present invention has been described in some detail, for clarity of understanding and by way of example, a variety of adaptations, changes, and modifications will be obvious to those who are skilled in the art. Hence, the scope of the present invention is limited solely by the following claims.

1. A catheter for delivering a contraceptive device within a fallopian tube, the catheter comprising:
an elongate tubular catheter body having a proximal portion adjacent a proximal end, a distal portion adjacent a distal end, and at least one lumen; and
at least one coil disposed along the catheter body nearer the distal end than the proximal end and encircling the lumen.

2. A catheter as in claim 1, wherein the distal portion of the catheter body is more flexible towards the distal end of the catheter body than towards the proximal end.

3. A catheter as in claim 2, wherein the distal portion of the catheter body comprises multiple layers, and the at least one coil comprises one of the layers.

4. A catheter as in claim 3, wherein the multiple layers comprise:
an inner layer;
a middle layer; and
an outer layer.

5. A catheter as in claim 4, wherein the middle layer comprises the coil.

6. A catheter as in claim 5, wherein the coil comprises at least one material selected from the group consisting of Nitinol®, stainless steel, titanium and a polymer.

7. A catheter as in claim 4, wherein the inner layer comprises at least one material selected from the group consisting of Teflon®, etched polytetraflouroethylene and a fluoropolymer.

8. A catheter as in claim 4, wherein the outer layer comprises at least one polyurethane material.

9. A catheter as in claim 8, wherein the polyurethane material comprises Carbothane.

10. A catheter as in claim 2, wherein the distal portion comprises:
a first segment; and
at least a second segment distal to the first segment,
wherein the second segment is more flexible than the first segment.

11. A catheter as in claim 10, further comprising a third segment distal to the second segment, wherein the third segment is more flexible than the second segment.

12. A catheter as in claim 11, wherein the distal portion comprises:
an inner layer;
a middle layer; and
an outer layer.

13. A catheter as in claim 12, wherein the middle layer comprises the coil and the outer layer comprises at least one polyurethane material.

14. A catheter as in claim 13, wherein the at least one polyurethane material comprises at least two polyurethane materials for conferring varying levels of flexibility to the distal portion.

15. A catheter as in claim 13, wherein the at least one polyurethane material has an increasing amount of flexibility from a proximal end of the distal portion to a distal end of the distal portion.

16. A catheter as in claim 1, wherein a pitch of the at least one coil is approximately 0.030 cm.

17. A catheter as in claim 1, wherein the distal portion of the catheter body has a length of between about 1.2 cm and about 2.0 cm.

18. A catheter as in claim 17, wherein the at least one coil has a length of between about 1.6 cm and about 2.4 cm.

19. A catheter as in claim 18, wherein the at least one coil extends through at least part of the distal portion of the catheter body and at least part of the proximal portion of the catheter body.

20. A catheter as in claim 19, wherein a distal end of the proximal portion of the catheter body overlaps a proximal end of the distal portion of the catheter body.

21. A catheter as in claim 18, wherein the length of the catheter body is between about 43 cm and about 50 cm.

22. A catheter as in claim 1, wherein an inner diameter of the proximal portion of the catheter body is smaller near the distal end of the catheter body than near the proximal end.

23. A catheter as in claim 1, wherein the proximal portion of the catheter body comprises at least one polyether block amide.

24. A catheter as in claim 1, wherein the proximal portion of the catheter body includes at least one visualization marker near the distal portion for enhancing visualization of a proximal-most end of the distal portion.

25. A catheter as in claim 24, wherein the visualization marker comprises at least one radiopaque material.

26. A catheter for delivering a contraceptive device within a fallopian tube, the catheter comprising:
an elongate tubular catheter body having a proximal portion adjacent a proximal end, a distal portion adjacent a distal end, and at least one lumen, wherein the distal portion is more flexible towards the distal end than towards the proximal end; and
at least one coil disposed along the catheter body nearer the distal end than the proximal end and encircling the lumen.

27. A catheter for delivering a contraceptive device within a fallopian tube, the catheter comprising:
an elongate tubular catheter body having a proximal portion adjacent a proximal end, a distal portion of between about 1.2 cm and about 2.0 cm adjacent a distal end, and at least one lumen, wherein the distal portion is more flexible towards the distal end than towards the proximal end; and
at least one coil disposed along the catheter body nearer the distal end than the proximal end and encircling the lumen.

28. A system for delivering a contraceptive device within a fallopian tube, the system comprising:
a catheter comprising:
an elongate tubular catheter body having a proximal portion adjacent a proximal end, a distal portion adjacent a distal end, and at least one lumen; and
at least one coil disposed along the catheter body nearer the distal end than the proximal end and encircling the lumen;
a contraceptive device releasably disposed at least partially within the lumen of the catheter near the distal portion; and
a deployment member in detachable engagement with the contraceptive device for deploying the contraceptive device from the catheter.

29. A system as in claim 28, wherein the distal portion of the catheter body is more flexible towards the distal end of the catheter body than towards the proximal end.

30. A system as in claim 29, wherein the distal portion of the catheter body comprises multiple layers, and the at least one coil comprises one of the layers.

31. A system as in claim 30, wherein the multiple layers comprise:
an inner layer;
a middle layer; and
an outer layer.

32. A system as in claim 31, wherein the middle layer comprises the coil.

33. A system as in claim 32, wherein the coil comprises at least one material selected from the group consisting of Nitinol®, stainless steel, titanium and a polymer.

34. A system as in claim 31, wherein the inner layer comprises at least one material selected from the group consisting of Teflon®, etched polytetraflouroethylene and a fluoropolymer.

35. A system as in claim 31, wherein the outer layer comprises at least one polyurethane material.

36. A system as in claim 35, wherein the polyurethane material comprises Carbothane.

37. A system as in claim 29, wherein the distal portion comprises:
a first segment; and
at least a second segment distal to the first segment,
wherein the second segment is more flexible than the first segment.

38. A system as in claim 37, further comprising a third segment distal to the second segment, wherein the third segment is more flexible than the second segment.

39. A system as in claim 38, wherein the distal portion comprises:
an inner layer;
a middle layer; and
an outer layer.

40. A system as in claim 39, wherein the middle layer comprises the coil and the outer layer comprises at least one polyurethane material.

41. A system as in claim 40, wherein the at least one polyurethane material comprises at least two polyurethane materials for conferring varying levels of flexibility to the distal portion.

42. A system as in claim 40, wherein the at least one polyurethane material has an increasing amount of flexibility from a proximal end of the distal portion to the distal end of the distal portion.

43. A system as in claim 28, wherein the proximal portion of the catheter body includes at least one visualization marker near the distal portion for enhancing visualization of a proximal-most end of the distal portion.

44. A system as in claim 43, wherein the visualization marker comprises at least one radiopaque material.

45. A method for making a catheter for delivery of a contraceptive device within a fallopian tube, the method comprising:
forming a distal portion of the catheter, comprising:
positioning a helical coil around an inner tubular member; and
placing at least one outer layer of material over the helical coil and the inner tubular member; and
coupling a proximal portion of the catheter with the distal portion of the catheter.

46. A method as in claim 45, wherein the inner tubular member comprises a metal selected from the group consisting of Teflon®, etched polytetrafluoroethylene and a fluoropolymer.

47. A method as in claim 45, wherein the helical coil comprises a metal selected from the group consisting of Nitinol®, stainless steel, titanium and a polymer.

48. A method as in claim 45, wherein the outer material comprises at least one polyurethane material.

49. A method as in claim 45, wherein coupling comprises overlapping a distal end of the proximal portion of the catheter with a proximal end of the distal portion of the catheter.

50. A method as in claim 49, wherein coupling further comprises heat welding the proximal portion to the distal portion.

51. A method as in claim 45, further comprising coupling a first segment of the outer material with at least a second segment of the outer material.

52. A method as in claim 51, further comprising coupling a third segment of the outer material with the second segment.

53. A method as in claim 53, wherein the first segment of the outer material has greater flexibility that the second segment, the second segment has greater flexibility than the third segment, and the third segment is coupled with the proximal portion of the catheter.

54. A method as in claim 46, wherein the distal portion of the catheter is between about 1.2 cm and about 2.0 cm, the coil is between about 1.6 cm and about 2.4 cm, and the catheter is between about 43 cm and about 50 cm in length.
